(19) United States
(12) Patent Application Publication
(10) Pub. No.: US 2025/0130416 A1
(43) Pub. Date: Apr. 24, 2025

(54) ILLUMINATION OPTICAL SYSTEM AND ENDOSCOPE

(71) Applicant: FUJIFILM CORPORATION, Tokyo (JP)

(72) Inventor: Keisuke HARADA, Saitama (JP)

(21) Appl. No.: 18/905,155

(22) Filed: Oct. 3, 2024

(30) Foreign Application Priority Data
Oct. 18, 2023 (JP) 2023-179876

(51) Int. Cl. G02B 23/24; G02B 9/10

(52) U.S. Cl. CPC G02B 23/243; G02B 9/10; G02B 23/2469

(57) ABSTRACT

An illumination optical system that is disposed at a distal end of a light guide of an endoscope, the illumination optical system including only two or less lenses as lenses, wherein: the illumination optical system has negative optical power as a whole, a first lens surface closest to the light guide includes a concave surface that is provided at a center portion thereof and a light diffusion surface that is provided outside the concave surface in a radial direction. The illumination optical system satisfies a predetermined conditional expression.

### CROSS-REFERENCE TO RELATED APPLICATIONS

This application claims priority from Japanese Application No. 2023-179876, filed on Oct. 18, 2023, the entire disclosure of which is incorporated herein by reference.

### BACKGROUND

### Technical Field

A technology of the present disclosure relates to an illumination optical system and an endoscope.

### Related Art

In the related art, for example, illumination optical systems disclosed in JP1982-097508A (JP-S57-097508A), JP3060245B, and JP1993-142485A (JP-H05-142485A) are known as an illumination optical system that is disposed at a distal end portion of an insertion part of an endoscope and is used to illuminate an object to be examined.

In recent years, there has been a demand for an illumination optical system that is formed to be small, has a wide light distribution angle, and has good transmission efficiency.

### SUMMARY

The present disclosure provides an illumination optical system that is formed to be small, has a wide light distribution angle, and has good transmission efficiency, and an endoscope comprising the illumination optical system.

A first illumination optical system of the present disclosure is an illumination optical system that is disposed at a distal end of a light guide of an endoscope and comprises only two or less lenses as lenses, in which the illumination optical system has negative optical power as a whole, a first lens surface closest to the light guide includes a concave surface that is provided at a center portion thereof and a light diffusion surface that is provided outside the concave surface in a radial direction, and, in a case where an absolute value of a paraxial curvature radius of the concave surface is denoted by R, a distance on an optical axis between an emission end surface of the light guide and the first lens surface is denoted by D, and a diameter of the emission end surface of the light guide is denoted by G, Conditional Expression (1) is satisfied,

$$0.6 < \frac{2 \sqrt{{R}^{2} - {( R - D )}^{2}}}{G} < 0.85 \qquad ( 1 )$$

A second illumination optical system of the present disclosure is an illumination optical system that is disposed at a distal end of a light guide of an endoscope and comprises only two or less lenses as lenses, in which the illumination optical system has negative optical power as a whole, a first lens surface closest to the light guide includes a concave surface that is provided at a center portion thereof and a light diffusion surface that is provided outside the concave surface in a radial direction, and, in a case where an absolute value of a paraxial curvature radius of the concave surface is denoted by R, a distance on an optical axis between an emission end surface of the light guide and the first lens surface is denoted by D, a diameter of the emission end surface of the light guide is denoted by G, and a focal length of the illumination optical system is denoted by f, Conditional Expression (2) is satisfied,

$$0.1 < \frac{2 ( \sqrt{{R}^{2} - {( R - D )}^{2}} ) - G}{f} < 1. \qquad ( 2 )$$

Hereinafter, the first illumination optical system and the second illumination optical system of the present disclosure will be collectively referred to as an illumination optical system of the present disclosure.

It is preferable that Conditional Expressions (3) and (4) are satisfied in the illumination optical system of the present disclosure.

$$0.2 < R / G < 0.6 \qquad ( 3 )$$

$$1.8 < \frac{2 \sqrt{{R}^{2} - {( R - D )}^{2}}}{D} < 4 \qquad ( 4 )$$

In a case where a diameter of a core in a direction perpendicular to the optical axis is denoted by C in a case where the lens closest to the light guide has a core-clad structure in which the core is coated with a clad and an outer diameter of the first lens surface is denoted by C in a case where the lens closest to the light guide does not have the core-clad structure, it is preferable that Conditional Expressions (5) and (6) are satisfied in the illumination optical system of the present disclosure.

$$0.8 < C / G < 1.5 \qquad ( 5 )$$

$$0.4 < \frac{2 \sqrt{{R}^{2} - {( R - D )}^{2}}}{C} < 0.8 \qquad ( 6 )$$

In a case where a refractive index of a core with respect to a d line is denoted by Nd1 in a case where the lens closest to the light guide has a core-clad structure in which the core is coated with a clad and a refractive index of the lens closest to the light guide with respect to the d line is denoted by Nd1 in a case where the lens closest to the light guide does not have the core-clad structure, in a case where a distance on the optical axis between the first lens surface and a surface closest to an irradiation target is denoted by L and a focal length of the illumination optical system is denoted by f, it is preferable that Conditional Expression (7) is satisfied in the illumination optical system of the present disclosure.

$$- 1 < L / ( Nd 1 \times f ) < - 0.22 \qquad ( 7 )$$

It is preferable that Conditional Expression (1-1) is satisfied in the first illumination optical system of the present disclosure.

$$0.7 < \frac{2 \sqrt{{R}^{2} − {( R − D )}^{2}}}{G} < 0.83 \qquad ( 1 - 1 )$$

It is preferable that Conditional Expression (2-1) is satisfied in the second illumination optical system of the present disclosure.

$$0.2 < \frac{2 ( \sqrt{{R}^{2} − {( R − D )}^{2}} ) - G}{f} < 0.6 \qquad ( 2 - 1 )$$

It is preferable that at least one of Conditional Expression (3-1) or (4-1) is satisfied in the illumination optical system of the present disclosure.

$$0.3 < R / G < 0.5 \qquad ( 3 - 1 )$$

$$2 < \frac{2 \sqrt{{R}^{2} − {( R − D )}^{2}}}{D} < 3.8 \qquad ( 4 - 1 )$$

It is preferable that at least one of Conditional Expression (5-1) or (6-1) is satisfied in the illumination optical system of the present disclosure.

$$1. < C / G < 1.4 \qquad ( 5 - 1 )$$

$$0.45 < \frac{2 \sqrt{{R}^{2} − {( R − D )}^{2}}}{C} < 0.75 \qquad ( 6 - 1 )$$

It is preferable that Conditional Expression (7-1) is satisfied in the illumination optical system of the present disclosure.

$$- 0.5 < L / ( Nd 1 \times f ) < - 0.21 \qquad ( 7 - 1 )$$

An endoscope of the present disclosure comprises the illumination optical system of the present disclosure.

“Consisting of” and “consist of” in this specification may intend to include: a lens substantially not having optical power; optical elements other than the lens, such as a stop, a filter, and a cover glass; a lens flange; a lens barrel; and the like in addition to mentioned components.

“Single lens” means one lens that is not cemented. The number of lenses described above is the number of lenses as components. For example, it is assumed that the number of lenses in a cemented lens in which a plurality of single lenses made of different materials are cemented is represented by the number of single lenses forming the cemented lens. However, a compound aspherical lens (that is, a lens of which a lens (for example, a spherical lens) and an aspherical film formed on the lens are integrally formed and which functions as one aspherical lens as a whole) is not regarded as a cemented lens, but is regarded as one lens.

A sign of the optical power of a lens that includes an aspherical surface, the curvature radius of a surface of the lens, and the shape of a surface of the lens are considered in a paraxial region unless otherwise specified. A sign of a curvature radius is positive for a surface with a convex shape toward a light guide side and is negative for a surface with a convex shape toward an irradiation target side.

“Focal length” used in Conditional Expression is a paraxial focal length. Unless otherwise specified, the “distance on the optical axis” used in Conditional Expression is a geometrical distance. A value used in Conditional Expression is a value with respect to the d line (a wavelength of 587.56 nm (nanometers)).

According to the present disclosure, it is possible to provide an illumination optical system that is formed to be small, has a wide light distribution angle, and has good transmission efficiency, and an endoscope comprising the illumination optical system.

### BRIEF DESCRIPTION OF THE DRAWINGS

FIG. 1 is a cross-sectional view showing a configuration of an illumination optical system according to an embodiment corresponding to an illumination optical system of Example 1.

FIG. 2 is a diagram illustrating a light diffusion surface.

FIG. 3 is a diagram illustrating the light diffusion surface.

FIG. 4 is a diagram illustrating a comparative example in which a light diffusion surface is not provided.

FIG. 5 is a cross-sectional view showing a configuration and optical paths of the illumination optical system of Example 1.

FIG. 6 is a cross-sectional view showing the configuration and the optical paths of the illumination optical system of Example 1.

FIG. 7 is a cross-sectional view showing a configuration and optical paths of an illumination optical system of Example 2.

FIG. 8 is a cross-sectional view showing the configuration and the optical paths of the illumination optical system of Example 2.

FIG. 9 is a cross-sectional view showing a configuration and optical paths of an illumination optical system of Example 3.

FIG. 10 is a cross-sectional view showing the configuration and the optical paths of the illumination optical system of Example 3.

FIG. 11 is a cross-sectional view showing a configuration and optical paths of an illumination optical system of Example 4.

FIG. 12 is a cross-sectional view showing the configuration and the optical paths of the illumination optical system of Example 4.

FIG. 13 is a cross-sectional view showing a configuration and optical paths of an illumination optical system of Example 5.

FIG. 14 is a cross-sectional view showing the configuration and the optical paths of the illumination optical system of Example 5.

FIG. 15 is a cross-sectional view showing a configuration and optical paths of an illumination optical system of Example 6.

FIG. 16 is a cross-sectional view showing the configuration and the optical paths of the illumination optical system of Example 6.

FIG. 17 is a cross-sectional view showing a configuration and optical paths of an illumination optical system of Example 7.

FIG. 18 is a cross-sectional view showing the configuration and the optical paths of the illumination optical system of Example 7.

FIG. 19 is a cross-sectional view showing a configuration and optical paths of an illumination optical system of Example 8.

FIG. 20 is a cross-sectional view showing the configuration and the optical paths of the illumination optical system of Example 8.

FIG. 21 is a cross-sectional view showing a configuration and optical paths of an illumination optical system of Example 9.

FIG. 22 is a cross-sectional view showing the configuration and the optical paths of the illumination optical system of Example 9.

FIG. 23 is a diagram showing a schematic configuration of an endoscope according to an embodiment.

### DETAILED DESCRIPTION

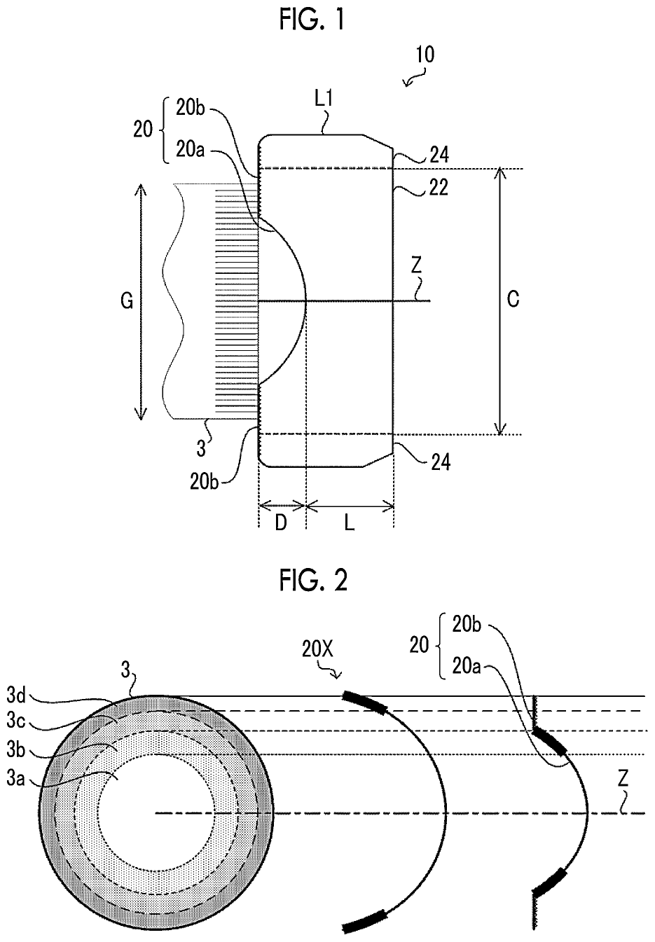

Embodiments of the present disclosure will be described in detail below with reference to the drawings. FIG. 1 is a cross-sectional view showing a configuration of an illumination optical system 10 according to an embodiment of the present disclosure. The illumination optical system 10 shown in FIG. 1 corresponds to Example 1 to be described later.

The illumination optical system 10 is an optical system that is disposed at a distal end of a light guide 3 of an endoscope. The light guide 3 consists of a bundle fiber in which a plurality of optical fibers are bundled, and emits light, which is emitted from a light source (not shown), to the illumination optical system 10. That is, the light emitted from the light source is incident on the illumination optical system 10 via the light guide 3, is emitted from the illumination optical system 10, and serves as illumination light. In a case where the illumination optical system 10 is disposed at a distal end of an insertion part of the endoscope, an irradiation target (not shown), which is an object to be observed, is illuminated with the illumination light. FIG. 1 is a cross-sectional view which includes an optical axis Z and in which a left side is a light source side and a right side is an irradiation target side.

The illumination optical system 10 according to the embodiment of the present disclosure comprises only two or less lenses as lenses, and has negative optical power as a whole. Here, each of the two or less lenses comprised in the illumination optical system 10 may be a cemented lens in which two lenses are cemented to each other. Since the number of lenses of the illumination optical system 10 is set to two or less and the illumination optical system 10 is adapted to have negative optical power as a whole, it is easy to increase a light distribution angle while achieving a reduction in size. For example, the illumination optical system 10 shown in FIG. 1 consists of one negative lens L1. The illumination optical system 10 may include an optical member that does not have optical power, for example, a plane-parallel plate.

In the illumination optical system 10, a first lens surface 20 closest to the light guide 3 includes a concave surface 20a that is provided at a center portion thereof and a light diffusion surface 20b that is provided outside the concave surface 20a in a radial direction. Since the center portion of the first lens surface 20 is formed of the concave surface 20a, it is easy to increase a light distribution angle while achieving a reduction in size. Since the light diffusion surface 20b is provided outside the concave surface 20a of the first lens surface 20 in the radial direction, it is easy to improve the transmission efficiency of light and to maintain a wide light distribution angle (details will be described later). The light diffusion surface 20b is formed by, for example, polishing performed on the surface of the lens.

In the illumination optical system 10, the lens closest to the light guide 3 may be a lens having a core-clad structure in which a core 22 is coated with a clad 24 (a lens consisting of a so-called single fiber). According to the core-clad structure, light incident on a boundary surface between the core 22 and the clad 24, which have different refractive indexes, is totally reflected and travels to the periphery of a visual field on an opposite side. Accordingly, even if the light is refracted by the concave surface 20a or diffused by the light diffusion surface 20b, the leakage of light from the outer periphery of the lens can be suppressed. Further, in a case where a lens surface opposite to the concave surface 20a is chamfered, light passing through the chamfered portion may be absorbed. However, it is possible to make light not to pass through the chamfered portion using total reflection caused by the core-clad structure. That is, in a case where the lens closest to the light guide 3 has the core-clad structure, the transmission efficiency of light can be improved.

For example, the lens LI shown in FIG. 1 has the core-clad structure that includes the core 22 and the clad 24. In FIG. 1, a boundary between the core 22 and the clad 24 is shown by a dotted line. The concave surface 20a is formed on the core 22 on the first lens surface 20 closest to the light guide 3. The light diffusion surface 20b is formed to extend over both the core 22 and the clad 24. For example, the light diffusion surface 20b may be formed only on the core 22 and may not be formed on the clad 24.

The light diffusion surface 20b will be described in detail with reference to FIGS. 2 to 4.

FIG. 2 is a diagram showing a correspondence relationship between radial positions of an emission end surface of the light guide 3 and the first lens surface 20. A lens surface 20X that is formed of only a concave surface and does not include a light diffusion surface is also shown in FIG. 2 as a comparative example. Further, in FIG. 2, for the sake of description, the emission end surface of the light guide 3 is divided into four equal regions 3a to 3d so that the regions have the same area. That is, the amounts of light emitted from the respective regions 3a to 3d are the same.

In a case where a diameter of the concave surface is equal to or larger than a diameter of the emission end surface of the light guide 3 as in the lens surface 20X of the comparative example, light emitted from an outer region (for example, a region 3d) of the emission end surface of the light guide 3 diverges when passing through the concave surface, which causes light leak. Even if, for example, the core-clad structure is used to suppress this light leak, a sufficient effect may not be obtained due to factors such as the spread angle of the light emitted from the light guide 3.

Further, since a change in the depth of the concave surface is steep toward the outer periphery, an area of the concave surface through which the light passes is increased. For example, an area of the lens surface 20X through which light emitted from the inner region 3a of the emission end surface of the light guide 3 passes is smaller than an area of the lens surface 20X through which the light emitted from the outer region 3d of the emission end surface of the light guide 3 passes. Therefore, an influence of light leak, the absorption of light caused by the chamfered portion, or the like on transmission efficiency is more significant at an outer peripheral portion (for example, a portion shown by a thick line in FIG. 2) of the concave surface than at a central portion thereof.

In the illumination optical system 10 according to the present embodiment, the light diffusion surface 20b is formed on a flat surface, which extends radially outward from the concave surface 20a, of the first lens surface 20 closest to the light guide 3. That is, since the light diffusion surface 20b is formed outside the outer peripheral portion of the concave surface 20a that is likely to cause the transmission efficiency of light to be reduced, it is possible to suppress a reduction in the transmission efficiency of light.

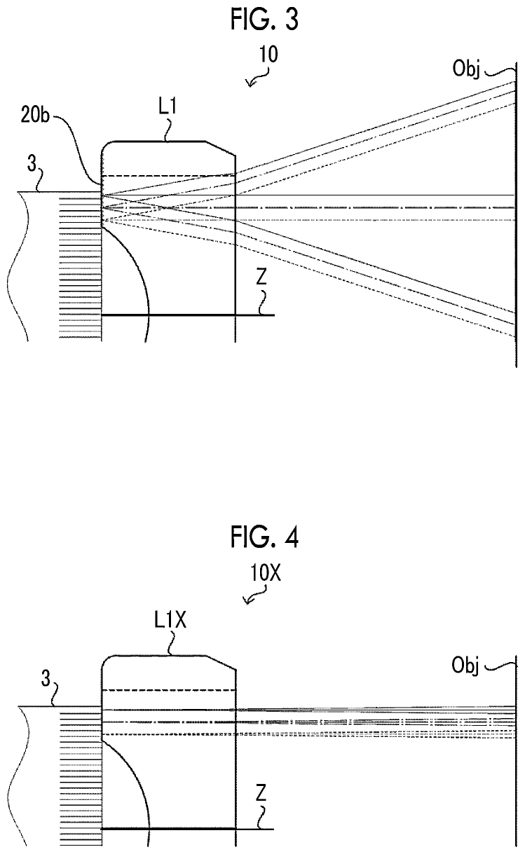

FIG. 3 is a diagram showing a state of rays that are emitted from the emission end surface of the light guide 3, are incident on the light diffusion surface 20b of the illumination optical system 10 shown in FIG. 1, and are emitted from the illumination optical system 10. FIG. 4 is a comparative example of FIG. 3, and is a diagram showing a state of rays in an illumination optical system 10X in which the lens L1 of the illumination optical system 10 shown in FIG. 1 is temporarily replaced with a lens LIX not having a light diffusion surface 20b. In FIGS. 3 and 4, rays incident on the lens L1 or the lens LIX from three points on the emission end surface of the light guide 3 are shown by a solid line, a one-dot chain line, and a dotted line, respectively.

As shown in FIG. 3, rays having passed through the light diffusion surface 20b are diffused and are projected onto an object surface Obj in a state where the spread of light is large. That is, since the rays are diffused by the light diffusion surface 20b as in the illumination optical system 10 according to the present embodiment, it is easy to maintain a wide light distribution angle.

Further, since the rays are diffused, it is possible to suppress that an image of the emission end surface of the light guide 3 is formed on the object surface Obj by the illumination optical system 10. Since the clad not emitting light, gaps between the fibers, and the core emitting light are arranged in the same plane on the emission end surface of the light guide 3 consisting of a bundle fiber, the emission end surface includes dark portions and bright portions. In a case where the image of the emission end surface of the light guide 3 is formed on the object surface Obj, a pattern of brightness and darkness (light distribution unevenness) is projected onto the object surface Obj. In a case where this projection image is clear, the observation of the object surface Obj (irradiation target) may be hindered. In a case where rays are diffused by the light diffusion surface 20b as in the present embodiment, the generation of the projection image of the pattern of brightness and darkness on the object surface Obj can be suppressed.

On the other hand, it can be seen that the degree of diffusion of light in the lens LIX not including the light diffusion surface 20b as shown in FIG. 4 is lower than that in the case of FIG. 3 and the light is projected onto the object surface Obj in a state where the spread of the light is small. That is, in the lens LIX not including the light diffusion surface 20b, as compared to the illumination optical system 10 according to the present embodiment, it is difficult to maintain a wide light distribution angle and the possibility of the generation of the projection image of the pattern of brightness and darkness (light distribution unevenness) on the object surface Obj is increased.

Next, preferable configurations and available configurations relating to Conditional Expressions of the illumination optical system according to the embodiment of the present disclosure will be described. In the description of the following conditional expressions, in order to avoid lengthy description, the same symbols will be used for terms having the same definition and repeated description of the symbols will be partially omitted. Further, in the following description, the “illumination optical system according to the embodiment of the present disclosure” will also be simply referred to as the “illumination optical system” in order to avoid lengthy description.

In a case where an absolute value of a paraxial curvature radius of the concave surface 20a is denoted by R, a distance on the optical axis Z between the emission end surface of the light guide 3 and the first lens surface 20 is denoted by D, and the diameter of the emission end surface of the light guide 3 is denoted by G, it is preferable that the illumination optical system 10 satisfies the following conditional expression (1). That is, in a case where a vertex of the concave surface 20a is positioned on the optical axis Z, D means a distance on the optical axis Z between the emission end surface of the light guide 3 and the vertex of the concave surface 20a. Since a corresponding value of Conditional Expression (1) is made to be larger than a lower limit, the concave surface 20a is not too small. Therefore, light distribution is not excessively narrowed, so that there is an advantage in increasing a light distribution angle. Since the corresponding value of Conditional Expression (1) is made to be smaller than an upper limit, the concave surface 20a is not too large. Therefore, transmission efficiency is not excessively reduced, so that there is an advantage in improving transmission efficiency. In order to obtain better characteristics, it is more preferable that the illumination optical system 10 satisfies the following conditional expression (1-1).

$$0.6 < \frac{2 \sqrt{{R}^{2} − {( R − D )}^{2}}}{G} < 0.85 \qquad ( 1 )$$

$$0.7 < \frac{2 \sqrt{{R}^{2} − {( R − D )}^{2}}}{G} < 0.83 \qquad ( 1 - 1 )$$

In a case where a focal length of the illumination optical system 10 is denoted by f, it is preferable that the illumination optical system 10 satisfies the following conditional expression (2). Since a corresponding value of Conditional Expression (2) is made to be larger than a lower limit, the concave surface 20a is not too small. Therefore, light distribution is not excessively narrowed, so that there is an advantage in increasing a light distribution angle. Since the corresponding value of Conditional Expression (2) is made to be smaller than an upper limit, the concave surface 20a is not too large. Therefore, transmission efficiency is not excessively reduced, so that there is an advantage in improving transmission efficiency. In order to obtain better characteristics, it is more preferable that the illumination optical system 10 satisfies the following conditional expression (2-1).

$$0.1 < \frac{2 ( \sqrt{{R}^{2} − {( R − D )}^{2}} ) - G}{f} < 1 \qquad ( 2 )$$

$$0.2 < \frac{2 ( \sqrt{{R}^{2} − {( R − D )}^{2}} ) - G}{f} < 0.6 \qquad ( 2 - 1 )$$

It is preferable that the illumination optical system 10 satisfies the following conditional expression (3). Since a corresponding value of Conditional Expression (3) is made to be larger than a lower limit, light does not excessively diverge. Therefore, it is possible to suppress the leakage of light from the outer periphery, so that it is possible to suppress a reduction in transmission efficiency. Since the corresponding value of Conditional Expression (3) is made to be smaller than an upper limit, light distribution is not excessively narrowed. Therefore, there is an advantage in increasing a light distribution angle. In order to obtain better characteristics, it is more preferable that the illumination optical system 10 satisfies the following conditional expression (3-1).

$$0.2 < R / G < 0.6 \qquad ( 3 )$$

$$0.3 < R / G < 0.5 \qquad ( 3 - 1 )$$

It is preferable that the illumination optical system 10 satisfies the following conditional expression (4). Since a corresponding value of Conditional Expression (4) is made to be larger than a lower limit, light does not excessively diverge. Therefore, the leakage of light from the outer periphery can be suppressed, so that a reduction in transmission efficiency can be suppressed. Since the corresponding value of Conditional Expression (4) is made to be smaller than an upper limit, light distribution is not excessively narrowed. Therefore, there is an advantage in increasing a light distribution angle. In order to obtain better characteristics, it is more preferable that the illumination optical system 10 satisfies the following conditional expression (4-1).

$$1.8 < \frac{2 \sqrt{{R}^{2} − {( R − D )}^{2}}}{D} < 4 \qquad ( 4 )$$

$$2 < \frac{2 \sqrt{{R}^{2} − {( R − D )}^{2}}}{D} < 3.8 \qquad ( 4 - 1 )$$

It is preferable that the illumination optical system 10 satisfies the following conditional expression (5). In a case where the lens L1 closest to the light guide 3 has the core-clad structure, a diameter of the core 22 in a direction perpendicular to the optical axis Z is denoted by C in Conditional Expression (5) and Conditional Expression (6) to be described later. On the other hand, in a case where the lens L1 closest to the light guide 3 does not have the core-clad structure, an outer diameter of the first lens surface 20 is denoted by C. Since a corresponding value of Conditional Expression (5) is made to be larger than a lower limit, the outer diameter of the lens L1 is not too small. Therefore, it is possible to suppress the leakage of light from the outer periphery while causing the light to sufficiently pass through the lens L1, so that it is possible to suppress a reduction in transmission efficiency. Since the corresponding value of Conditional Expression (5) is made to be smaller than an upper limit, the outer diameter of the lens L1 is not too large. Therefore, there is an advantage in achieving a reduction in size. In order to obtain better characteristics, it is more preferable that the illumination optical system 10 satisfies the following conditional expression (5-1).

$$0.8 < C / G < 1.5 \qquad ( 5 )$$

$$1. < C / G < 1.4 \qquad ( 5 - 1 )$$

It is preferable that the illumination optical system 10 satisfies the following conditional expression (6). Since a corresponding value of Conditional Expression (6) is made to be larger than a lower limit, the outer diameter of the lens L1 is not too large. Therefore, there is an advantage in achieving a reduction in size. Since the corresponding value of Conditional Expression (6) is made to be smaller than an upper limit, the outer diameter of the lens L1 is not too small. Therefore, it is possible to suppress the leakage of light from the outer periphery while causing the light to sufficiently pass through the lens L1, so that it is possible to suppress a reduction in transmission efficiency. In order to obtain better characteristics, it is more preferable that the illumination optical system 10 satisfies the following conditional expression (6-1).

$$0.4 < \frac{2 \sqrt{{R}^{2} − {( R − D )}^{2}}}{C} < 0.8 \qquad ( 6 )$$

$$0.45 < \frac{2 \sqrt{{R}^{2} − {( R − D )}^{2}}}{C} < 0.75 \qquad ( 6 - 1 )$$

In a case where a refractive index of the core 22 with respect to the d line is denoted by Nd1 in a case where the lens L1 closest to the light guide 3 has the core-clad structure and a refractive index of the lens L1 closest to the light guide with respect to the d line is denoted by Nd1 in a case where the lens L1 closest to the light guide 3 does not have the core-clad structure, in a case where a distance on the optical axis Z between the first lens surface 20 and a surface closest to the irradiation target is denoted by L, it is preferable that the illumination optical system 10 satisfies the following conditional expression (7). That is, L means the thickness of the entire illumination optical system 10 on the optical axis Z. Since a corresponding value of Conditional Expression (7) is made to be larger than a lower limit, a central thickness of the illumination optical system 10 is not too large. Therefore, there is an advantage in achieving a reduction in size. Since the corresponding value of Conditional Expression (7) is made to be smaller than an upper limit, the central thickness of the illumination optical system 10 is not too small. Therefore, the lens is less likely to break. In order to obtain better characteristics, it is more preferable that the illumination optical system 10 satisfies the following conditional expression (7-1).

$$- 1 < L / ( Nd 1 \times f ) < - 0.22 \qquad ( 7 )$$

$$- 0.5 < L / ( Nd 1 \times f ) < - 0.21 \qquad ( 7 - 1 )$$

The above-mentioned preferable configurations and available configurations including the configurations relating to Conditional Expressions can be arbitrarily combined, and it is preferable that the configurations are selectively employed as appropriate according to required specifications.

As an example, a preferred aspect of the illumination optical system according to the embodiment of the present disclosure is an illumination optical system that is disposed at a distal end of a light guide of an endoscope, comprises only two or less lenses as lenses, and has negative optical power as a whole. A first lens surface closest to the light guide includes a concave surface that is provided at a center portion thereof and a light diffusion surface that is provided outside the concave surface in a radial direction, and the illumination optical system satisfies Conditional Expression (1).

As another example, a preferred aspect of the illumination optical system according to the embodiment of the present disclosure is an illumination optical system that is disposed at a distal end of a light guide of an endoscope, comprises only two or less lenses as lenses, and has negative optical power as a whole. A first lens surface closest to the light guide includes a concave surface that is provided at a center portion thereof and a light diffusion surface that is provided outside the concave surface in a radial direction, and the illumination optical system satisfies Conditional Expression (2).

Next, examples of the illumination optical system according to the embodiment of the present disclosure will be described with reference to the drawings. Reference numeral given to each lens shown in a cross-sectional view of each example is used independently for each example in order to avoid complication of description and drawings caused by an increase in the number of digits of reference numeral. Accordingly, even though common reference numerals are given to components in the drawings of different examples, the components are not necessarily common.

### Example 1

A cross-sectional view showing a configuration of an illumination optical system of Example 1 is shown in FIG. 1 and a method of illustrating the cross-sectional view is as described above. Accordingly, repeated description will be partially omitted here. The illumination optical system of Example 1 consists of one negative lens L1. A first lens surface closest to the light guide 3 includes a concave surface that is provided at a center portion thereof and a light diffusion surface that is provided outside the concave surface in a radial direction. The lens L1 has a core-clad structure. An outline of the illumination optical system of Example 1 is as described above.

Basic lens data of the illumination optical system of Example 1 are shown in Table 1, and specifications thereof are shown in Table 2. The table of the basic lens data is described as follows. Surface numbers in a case where a surface closest to the light guide is set as a first surface and a surface number is increased by one toward an irradiation target are shown in a column of Sn. A curvature radius of each surface is shown in a column of R. A surface spacing on the optical axis between each surface and a surface, which is adjacent to an irradiation target side of the surface, is shown in a column of D. A refractive index of each lens with respect to the d line is shown in a column of Nd. An Abbe number of each lens with respect to the d line is shown in a column of vd. With regard to the lens having a core-clad structure, a refractive index Nd and an Abbe number vd of each of a core and a clad are shown.

Table 2 shows a diameter H of the concave surface of the first lens surface, a diameter G of the emission end surface of the light guide, a diameter C of the core of the lens L1 in a direction perpendicular to the optical axis Z, a maximum outer diameter Cm of the illumination optical system, and a focal length f of the illumination optical system with respect to the d line. Millimeters are used as a unit of length in the data of each table. However, since the optical system can be used even though the optical system is proportionally enlarged or reduced, other appropriate units can be used. Further, numerical values, which are rounded off to a predetermined place, are written in each table to be described below.

TABLE 1
Example 1

| Sn | R | D | Nd | vd |
|---|---|---|---|---|
| 1 | | 0.1700 | | |
| 2 | −0.3600 | 0.3100 | (Core) 1.744 | (Core) 42.6 |
| | | | (Clad) 1.515 | (Clad) 62.6 |
| 3 | ∞ | | | |

TABLE 2
Example 1

| | |
|---|---|
| H | 0.612 |
| G | 0.85 |
| C | 0.96 |
| Cm | 1.20 |
| f | −0.484 |

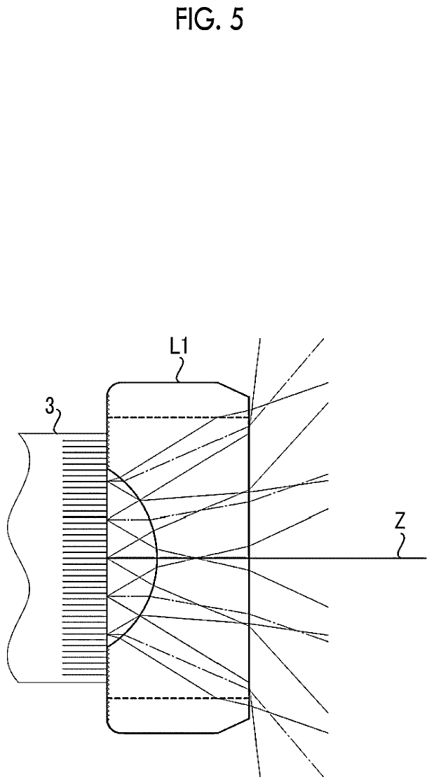

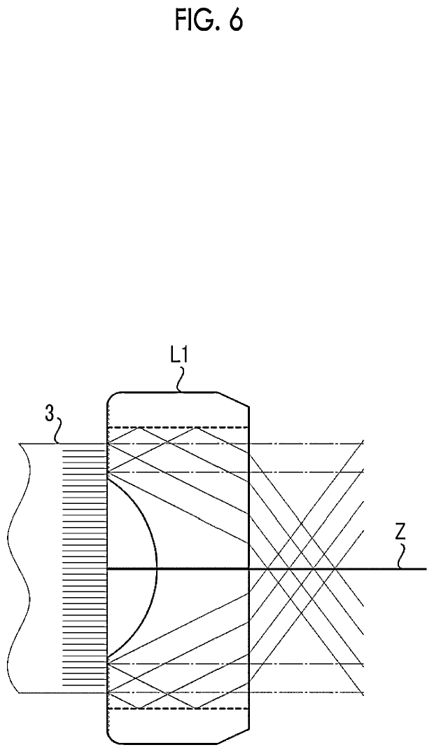

FIGS. 5 and 6 show cross-sectional views showing the configuration and optical paths of the illumination optical system of Example 1. The optical paths shown in FIGS. 5 and 6 are optical paths of rays in a case where a plurality of rays emitted from a plurality of points on the emission end surface of the light guide 3 are incident on the illumination optical system, FIG. 5 shows the optical paths of rays incident on the concave surface, and FIG. 6 shows the optical paths of rays incident on the light diffusion surface. Since methods of illustrating the cross-sectional views in FIGS. 5 and 6 are the same as that in FIG. 1, repeated description will be omitted here.

Symbols, meanings, description methods, and illustration methods of the respective data relating to Example 1 are the same as those in the following examples unless otherwise specified. Therefore, repeated description will be omitted in the following description.

### Example 2

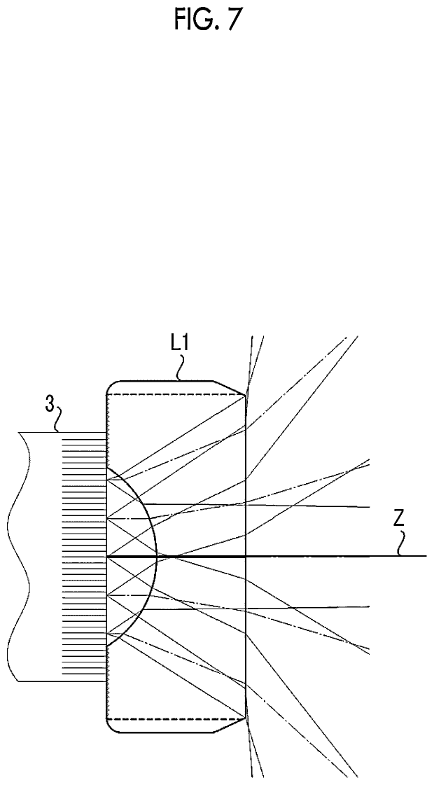

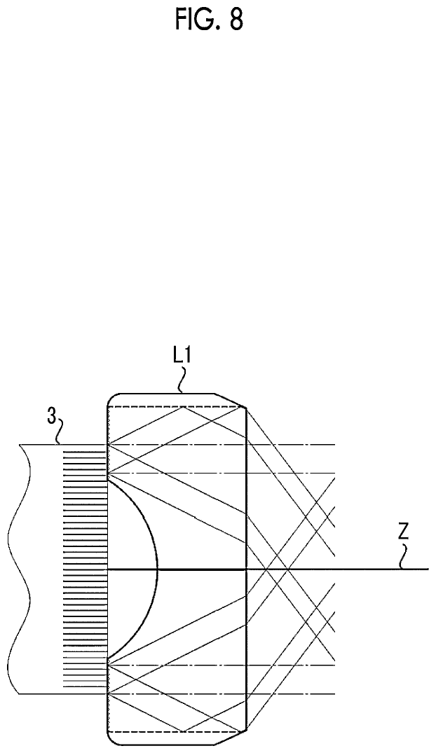

FIGS. 7 and 8 show cross-sectional views showing a configuration and optical paths of an illumination optical system of Example 2. FIG. 7 shows the optical paths of rays incident on a concave surface, and FIG. 8 shows the optical paths of rays incident on a light diffusion surface. The illumination optical system of Example 2 has the same configuration as the outline of the illumination optical system of Example 1. Basic lens data of the illumination optical system of Example 2 are shown in Table 3, and specifications thereof are shown in Table 4.

TABLE 3
Example 2

| Sn | R | D | Nd | vd |
|---|---|---|---|---|
| 1 | | 0.1700 | | |
| 2 | −0.3600 | 0.3000 | (Core) 1.744 | (Core) 42.6 |
| | | | (Clad) 1.515 | (Clad) 62.6 |
| 3 | ∞ | | | |

TABLE 4
Example 2

| | |
|---|---|
| H | 0.612 |
| G | 0.85 |
| C | 1.11 |
| Cm | 1.20 |
| f | −0.484 |

### Example 3

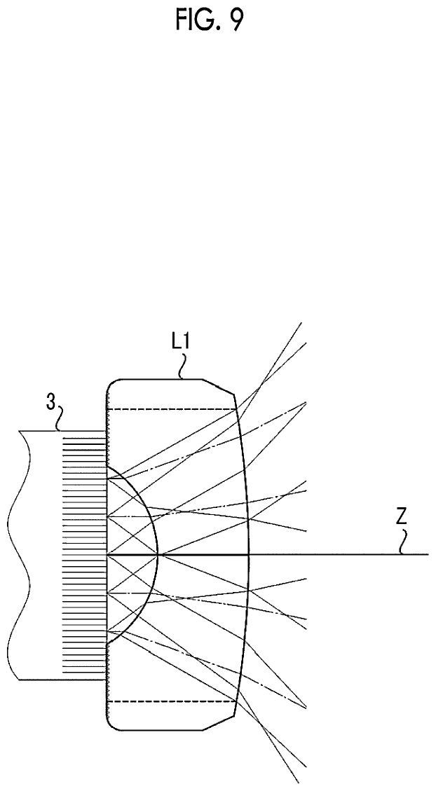

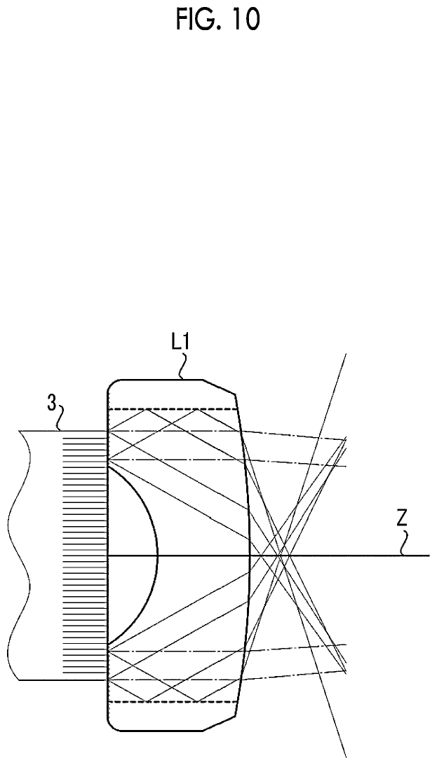

FIGS. 9 and 10 show cross-sectional views showing a configuration and optical paths of an illumination optical system of Example 3. FIG. 9 shows the optical paths of rays incident on a concave surface, and FIG. 10 shows the optical paths of rays incident on a light diffusion surface. The illumination optical system of Example 3 has the same configuration as the outline of the illumination optical system of Example 1. Basic lens data of the illumination optical system of Example 3 are shown in Table 5, and specifications thereof are shown in Table 6.

TABLE 5
Example 3

| Sn | R | D | Nd | νd |
|---|---|---|---|---|
| 1 | | 0.1700 | | |
| 2 | −0.3600 | 0.3100 | (Core) 1.620 | (Core) 36.3 |
| | | | (Clad) 1.367 | (Clad) 50.0 |
| 3 | −3.0487 | | | |

TABLE 6
Example 3

| | |
|---|---|
| H | 0.612 |
| G | 0.85 |
| C | 1.00 |
| Cm | 1.20 |
| f | −0.689 |

### Example 4

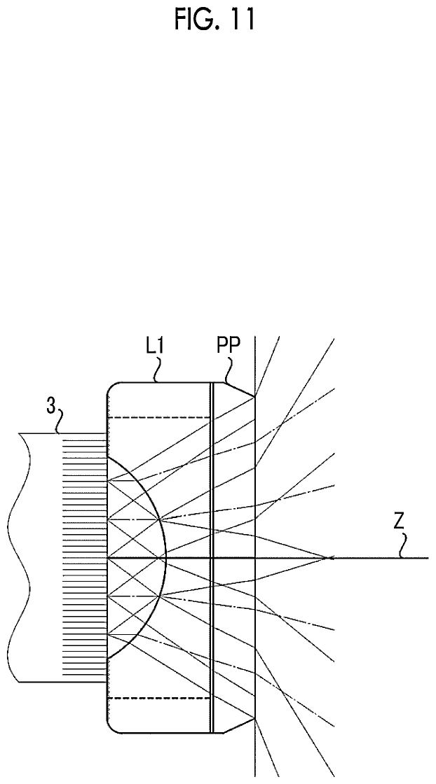

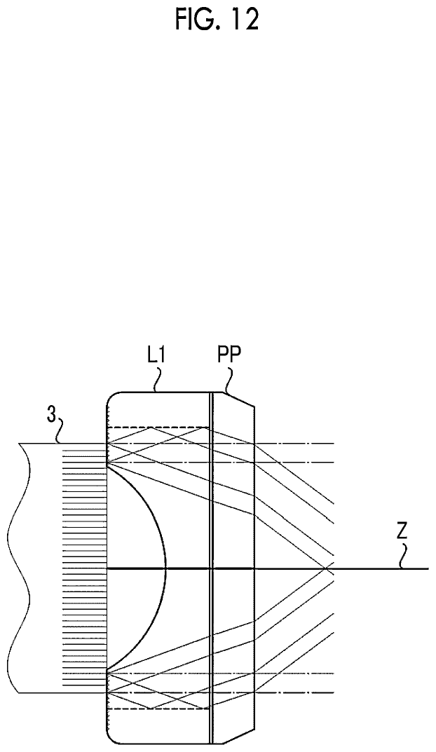

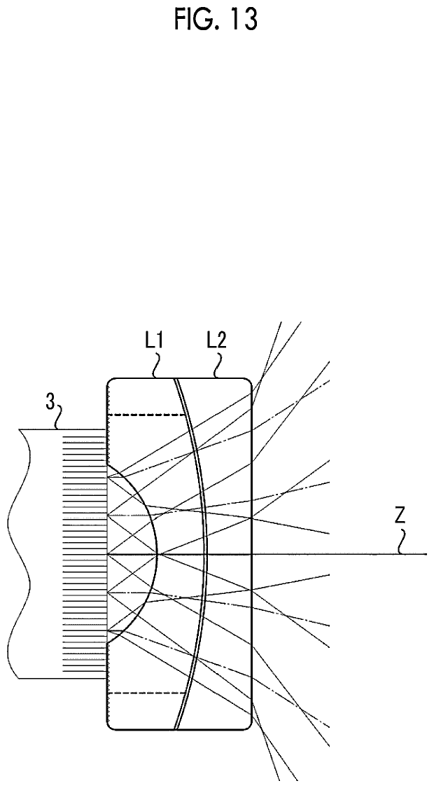

FIGS. 11 and 12 show cross-sectional views showing a configuration and optical paths of an illumination optical system of Example 4. FIG. 11 shows the optical paths of rays incident on a concave surface, and FIG. 12 shows the optical paths of rays incident on a light diffusion surface. The illumination optical system of Example 4 consists of a negative lens L1 and a plane-parallel plate PP, which has no optical power, in order from the light guide 3 to an irradiation target side. A first lens surface closest to the light guide 3 includes a concave surface that is provided at a center portion thereof and a light diffusion surface that is provided outside the concave surface in a radial direction. The lens L1 has a core-clad structure. Basic lens data of the illumination optical system of Example 4 are shown in Table 7, and specifications thereof are shown in Table 8. In Table 7, data of a cementing agent that cements the lens L1 and the plane-parallel plate PP is written in a row where Sn is 3.

TABLE 7
Example 4

| Sn | R | D | Nd | νd |
|---|---|---|---|---|
| 1 | | 0.2000 | | |
| 2 | −0.4000 | 0.1500 | (Core) 1.744 | (Core) 42.6 |
| | | | (Clad) 1.515 | (Clad) 62.6 |
| 3 | ∞ | 0.0100 | 1.56002 | 37.65 |
| 4 | ∞ | 0.1400 | 1.88299 | 40.78 |
| 5 | ∞ | | | |

TABLE 8
Example 4

| | |
|---|---|
| H | 0.693 |
| G | 0.85 |
| C | 0.96 |
| Cm | 1.20 |
| f | −0.538 |

### Example 5

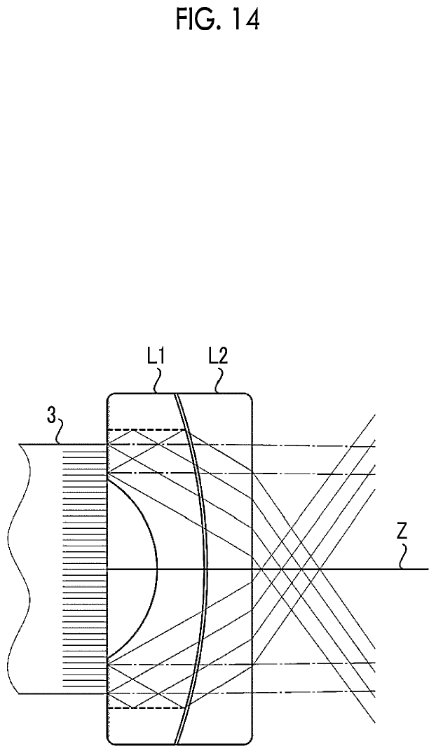

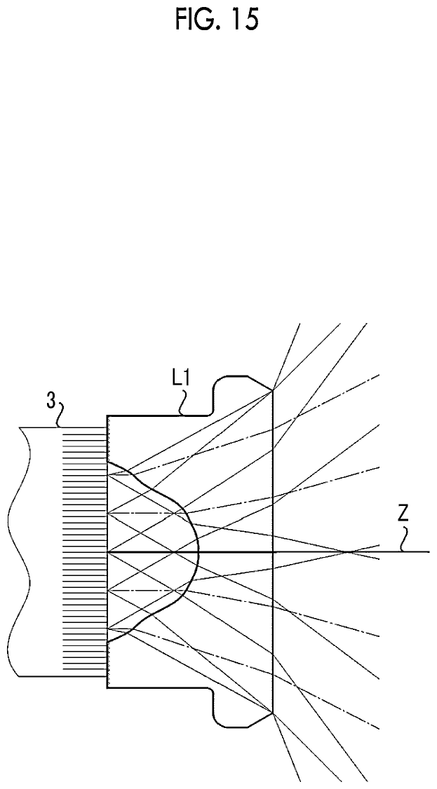

FIGS. 13 and 14 show cross-sectional views showing a configuration and optical paths of an illumination optical system of Example 5. FIG. 13 shows the optical paths of rays incident on a concave surface, and FIG. 14 shows the optical paths of rays incident on a light diffusion surface. The illumination optical system of Example 5 consists of a negative lens L1 and a negative lens L2 in order from the light guide 3 to an irradiation target side, and the lens L1 and the lens L2 are cemented to each other. A first lens surface closest to the light guide 3 includes a concave surface that is provided at a center portion thereof and a light diffusion surface that is provided outside the concave surface in a radial direction. The lens LI has a core-clad structure. The lens L2 does not have a core-clad structure. Basic lens data of the illumination optical system of Example 5 are shown in Table 9, and specifications thereof are shown in Table 10. In Table 9, data of a cementing agent that cements the lens L1 and the lens L2 is written in a row where Sn is 3.

TABLE 9
Example 5

| Sn | R | D | Nd | νd |
|---|---|---|---|---|
| 1 | | 0.1700 | | |
| 2 | −0.3600 | 0.1600 | (Core) 1.620 | (Core) 36.3 |
| | | | (Clad) 1.367 | (Clad) 50.0 |
| 3 | −1.8159 | 0.0100 | 1.56002 | 37.65 |
| 4 | −1.8159 | 0.1500 | 1.55919 | 53.90 |
| 5 | ∞ | | | |

TABLE 10
Example 5

| | |
|---|---|
| H | 0.612 |
| G | 0.85 |
| C | 0.95 |
| Cm | 1.20 |
| f | −0.594 |

### Example 6

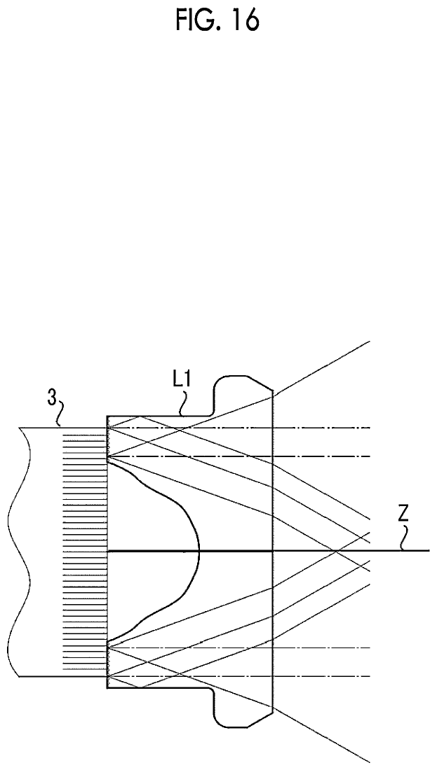

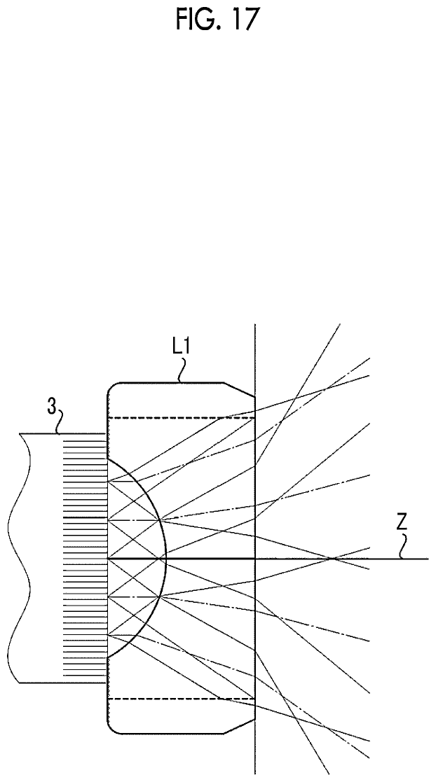

FIGS. 15 and 16 show cross-sectional views showing a configuration and optical paths of an illumination optical system of Example 6. FIG. 15 shows the optical paths of rays incident on a concave surface, and FIG. 16 shows the optical paths of rays incident on a light diffusion surface. The illumination optical system of Example 6 consists of one negative lens L1. A first lens surface closest to the light guide 3 includes a concave aspherical surface that is provided at a center portion thereof and a light diffusion surface that is provided outside the concave surface in a radial direction. Basic lens data of the illumination optical system of Example 6 are shown in Table 11, specifications thereof are shown in Table 12, and aspherical coefficients thereof are shown in Table 13. Since the lens L1 does not have a core-clad structure in Example 6, a diameter C of a core is omitted in Table 12. Further, an outer diameter Cn of the first lens surface is added to Table 12.

In the basic lens data, a reference sign * is attached to a surface number of the aspherical surface, and a value of a paraxial curvature radius is written in a field of a curvature radius of the aspherical surface. In Table 13, the surface number of the aspherical surface is written in a row of Sn and numerical values of aspherical coefficients of respective aspherical surfaces are written in rows of KA and Am. The “E±n” (n: an integer) in numerical values of the aspherical coefficients of Table 13 means “×10^{±n}”. KA and Am (m=4, 6, 8, . . . , 16) are aspherical coefficients in an aspherical equation that is represented by the following equation.

$$Z d = C \times {h}^{2} / \{ 1 + {( 1 - KA \times {C}^{2} \times {h}^{2} )}^{1 / 2} \} + \sum Am \times {h}^{m}$$

Here,
Zd is an aspherical surface depth (a length of a perpendicular to a plane, which is perpendicular to the optical axis Z and is in contact with the vertex of the aspherical surface, from a point on an aspherical surface having a height h),
h is a height (a distance between the optical axis Z and the lens surface),
C is an inverse of the paraxial curvature radius,
KA and Am are aspherical coefficients, and
Σ of the aspherical equation means the sum with respect to m.

TABLE 11
Example 6

| Sn | R | D | Nd | νd |
|---|---|---|---|---|
| 1 | | 0.3100 | | |
| 2* | −0.3250 | 0.2500 | 1.45850 | 67.82 |
| 3 | ∞ | | | |

TABLE 12
Example 6

| | |
|---|---|
| H | 0.615 |
| G | 0.85 |
| Cn | 0.93 |
| Cm | 1.20 |
| f | −0.709 |

TABLE 13
Example 6

| | |
|---|---|
| Sn | 2 |
| K | 1.00000000000E+00 |
| A4 | −3.07853636918E+02 |
| A6 | 3.41067643384E+04 |
| A8 | −1.67354510031E+06 |
| A10 | 4.05667742591E+07 |
| A12 | −5.14838795038E+08 |
| A14 | 3.29690809696E+09 |
| A16 | −8.42936182912E+09 |

### Example 7

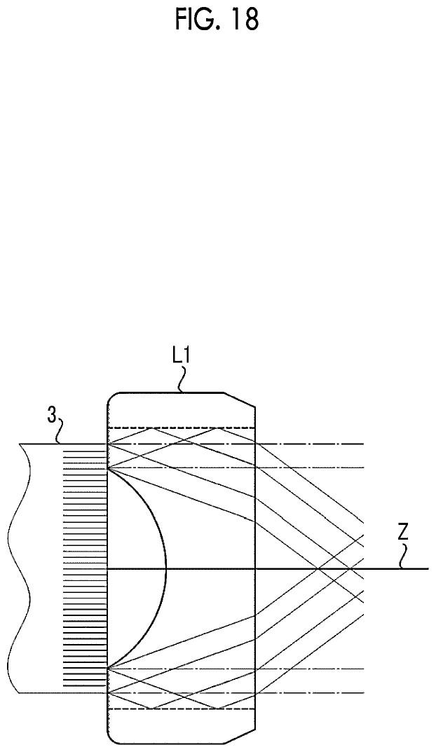

FIGS. 17 and 18 show cross-sectional views showing a configuration and optical paths of an illumination optical system of Example 7. FIG. 17 shows the optical paths of rays incident on a concave surface, and FIG. 18 shows the optical paths of rays incident on a light diffusion surface. The illumination optical system of Example 7 has the same configuration as the outline of the illumination optical system of Example 1. Basic lens data of the illumination optical system of Example 7 are shown in Table 14, and specifications thereof are shown in Table 15.

TABLE 14
Example 7

| Sn | R | D | Nd | νd |
|---|---|---|---|---|
| 1 | | 0.2000 | | |
| 2 | −0.3900 | 0.3000 | (Core) 1.744 | (Core) 42.6 |
| | | | (Clad) 1.515 | (Clad) 62.6 |
| 3 | ∞ | | | |

TABLE 15
Example 7

| | |
|---|---|
| H | 0.681 |
| G | 0.85 |
| C | 0.96 |
| Cm | 1.20 |
| f | −0.524 |

### Example 8

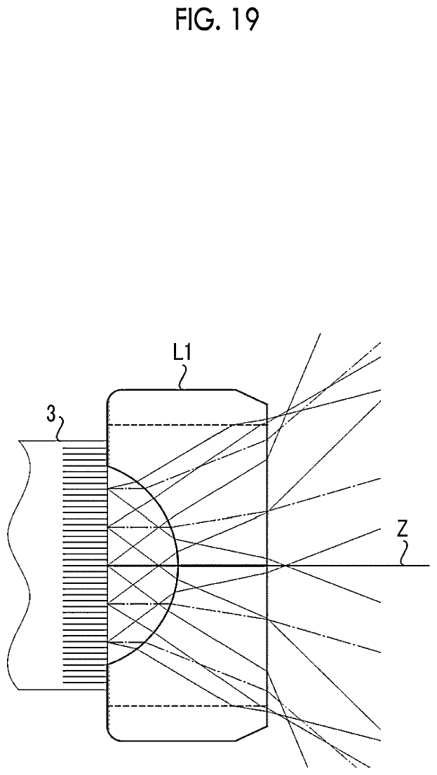

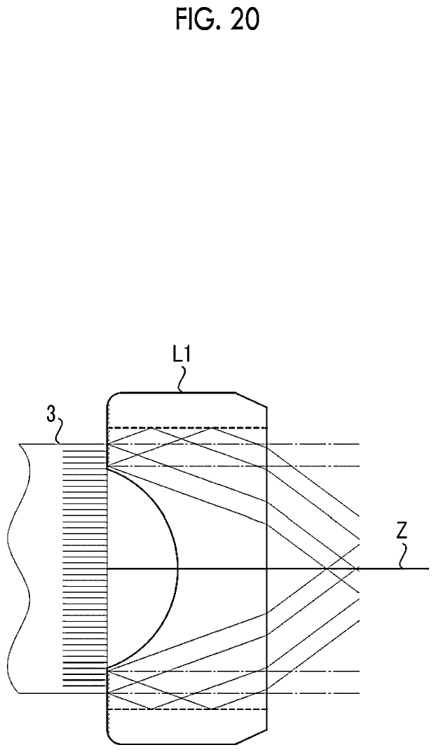

FIGS. 19 and 20 show cross-sectional views showing a configuration and optical paths of an illumination optical system of Example 8. FIG. 19 shows the optical paths of rays incident on a concave surface, and FIG. 20 shows the optical paths of rays incident on a light diffusion surface. The illumination optical system of Example 8 has the same configuration as the outline of the illumination optical system of Example 1. Basic lens data of the illumination optical system of Example 8 are shown in Table 16, and specifications thereof are shown in Table 17.

TABLE 16
Example 8

| Sn | R | D | Nd | νd |
|---|---|---|---|---|
| 1 | | 0.2400 | | |
| 2 | −0.3600 | 0.3000 | (Core) 1.744 | (Core) 42.6 |
| | | | (Clad) 1.515 | (Clad) 62.6 |
| 3 | ∞ | | | |

TABLE 17
Example 8

| | |
|---|---|
| H | 0.679 |
| G | 0.85 |
| C | 0.96 |
| Cm | 1.20 |
| f | −0.484 |

### Example 9

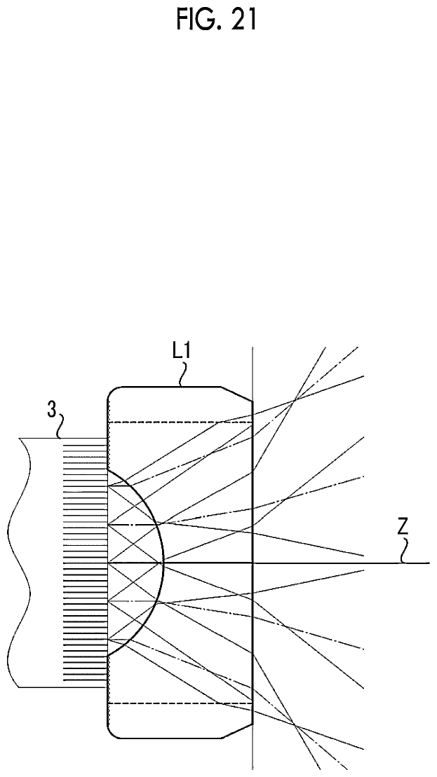

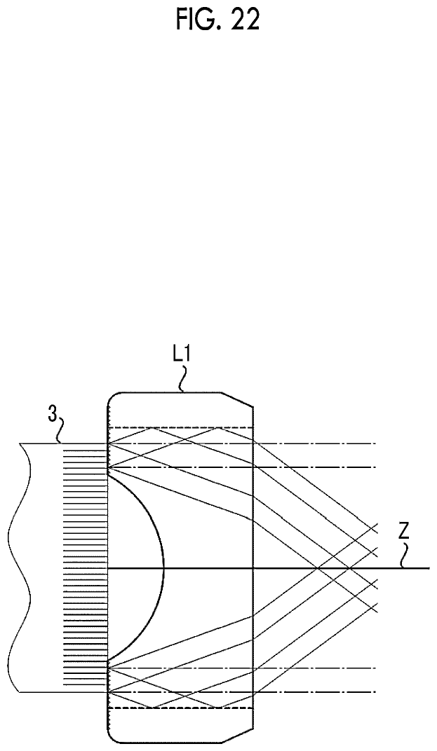

FIGS. 21 and 22 show cross-sectional views showing a configuration and optical paths of an illumination optical system of Example 9. FIG. 21 shows the optical paths of rays incident on a concave surface, and FIG. 22 shows the optical paths of rays incident on a light diffusion surface. The illumination optical system of Example 9 has the same configuration as the outline of the illumination optical system of Example 1. Basic lens data of the illumination optical system of Example 9 are shown in Table 18, and specifications thereof are shown in Table 19.

TABLE 18
Example 9

| Sn | R | D | Nd | νd |
|---|---|---|---|---|
| 1 | | 0.1900 | | |
| 2 | −0.3600 | 0.3000 | (Core) 1.744 | (Core) 42.6 |
| | | | (Clad) 1.515 | (Clad) 62.6 |
| 3 | ∞ | | | |

TABLE 19
Example 9

| | |
|---|---|
| H | 0.635 |
| G | 0.85 |
| C | 0.96 |
| Cm | 1.20 |
| f | −0.484 |

The corresponding values of Conditional Expressions (1) to (7) of the illumination optical systems of Examples 1 to 9 are shown in Table 20. Preferable ranges of Conditional Expressions may be set using the corresponding values of Examples shown in Table 20 as the upper limits or the lower limits of Conditional Expressions.

TABLE 20

| Expression | Conditional | Example | Example | Example | Example | Example | Example | Example | Example | Example |
|---|---|---|---|---|---|---|---|---|---|---|
| No. | Expression | 1 | 2 | 3 | 4 | 5 | 6 | 7 | 8 | 9 |
| (1) | $\frac{2 \sqrt{{R}^{2} - {( R - D )}^{2}}}{G}$ | 0.72 | 0.72 | 0.72 | 0.82 | 0.72 | 0.76 | 0.80 | 0.80 | 0.75 |
| (2) | $\frac{2 ( \sqrt{{R}^{2} - {( R - D )}^{2}} ) - G}{f}$ | 0.49 | 0.49 | 0.35 | 0.29 | 0.40 | 0.28 | 0.32 | 0.35 | 0.44 |
| (3) | R/G | 0.42 | 0.42 | 0.42 | 0.47 | 0.42 | 0.38 | 0.46 | 0.42 | 0.42 |
| (4) | $\frac{2 \sqrt{{R}^{2} - {( R - D )}^{2}}}{D}$ | 3.60 | 3.60 | 3.60 | 3.46 | 3.60 | 2.09 | 3.41 | 2.83 | 3.34 |
| (5) | C/G | 1.13 | 1.31 | 1.18 | 1.13 | 1.12 | 1.09 | 1.13 | 1.13 | 1.13 |
| (6) | $\frac{2 \sqrt{{R}^{2} - {( R - D )}^{2}}}{C}$ | 0.64 | 0.55 | 0.61 | 0.72 | 0.64 | 0.70 | 0.71 | 0.71 | 0.66 |
| (7) | L/(Nd1 × f) | −0.37 | −0.36 | −0.28 | −0.32 | −0.33 | −0.24 | −0.33 | −0.36 | −0.36 |

From the above-described data, it can be seen that all the illumination optical systems of Examples 1 to 9 are formed to be small, have a wide light distribution angle, and have good transmission efficiency.

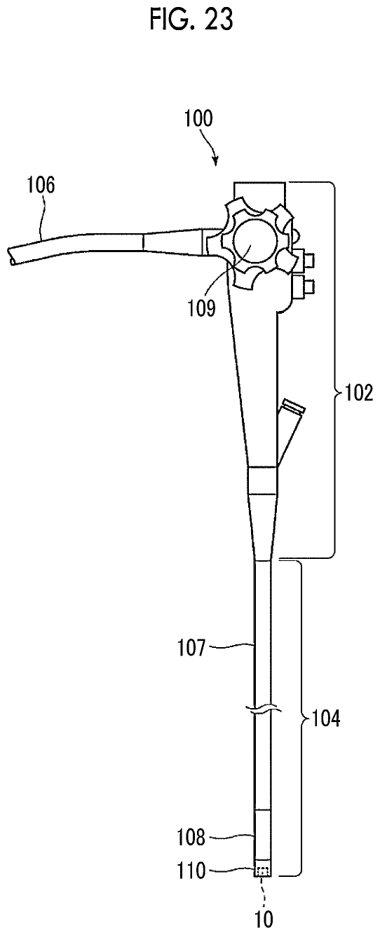

Next, an endoscope according to an embodiment of the disclosure will be described. FIG. 23 shows a diagram showing a schematic configuration of the entire endoscope according to the embodiment of the disclosure. The endoscope 100 shown in FIG. 23 mainly comprises an operation part 102, an insertion part 104, and a universal cord 106 that is to be connected to a connector part (not shown). A large portion of the insertion part 104 is a soft portion 107 that is bendable in any direction along an insertion path, a bendable portion 108 is connected to a distal end of the soft portion 107, and a distal end portion 110 is connected to a distal end of the bendable portion 108. The bendable portion 108 is provided to allow the distal end portion 110 to face in a desired direction, and can be operated to be bent by the rotational movement of bending operation knobs 109 provided on the operation part 102.

The illumination optical system 10 according to the embodiment of the present disclosure is provided in a distal end of the distal end portion 110. The illumination optical system 10 is schematically shown in FIG. 23. Since the endoscope according to the embodiment of the present disclosure comprises the illumination optical system according to the embodiment of the present disclosure, it is possible to perform observation using good-quality illumination light with a wide angle while reducing the size of a distal end portion of the insertion part 104.

The technology of the present disclosure has been described above using the embodiments and Examples. However, the technology of the present disclosure is not limited to the embodiments and Examples described above, and may be modified in various ways. For example, the curvature radius, the surface spacing, the refractive index, the Abbe number, the aspherical coefficient, and the like of each lens are not limited to the values shown in Examples described above, and may have other values.

With regard to the embodiments and Examples described above, the following supplementary notes will be further disclosed.

### Supplementary Note 1

An illumination optical system that is disposed at a distal end of a light guide of an endoscope, the illumination optical system comprising:
only two or less lenses as lenses,
wherein the illumination optical system has negative optical power as a whole,
a first lens surface closest to the light guide includes a concave surface that is provided at a center portion thereof and a light diffusion surface that is provided outside the concave surface in a radial direction, and
in a case where an absolute value of a paraxial curvature radius of the concave surface is denoted by R, a distance on an optical axis between an emission end surface of the light guide and the first lens surface is denoted by D, and a diameter of the emission end surface of the light guide is denoted by G, Conditional Expression (1) is satisfied,

$$0.6 < \frac{2 \sqrt{{R}^{2} − {( R − D )}^{2}}}{G} < 0.85 . \qquad ( 8 )$$

### Supplementary Note 2

The illumination optical system according to Supplementary note 1, wherein Conditional Expressions (3) and (4) are satisfied,

$$0.2 < R / G < 0.6 \qquad ( 3 )$$

$$1 \text{.8} < \frac{2 \sqrt{{R}^{2} − {( R − D )}^{2}}}{D} < 4. \qquad ( 4 )$$

### Supplementary Note 3

The illumination optical system according to Supplementary note 1 or 2,
wherein, in a case where a diameter of a core in a direction perpendicular to the optical axis is denoted by C in a case where the lens closest to the light guide has a core-clad structure in which the core is coated with a clad and an outer diameter of the first lens surface is denoted by C in a case where the lens closest to the light guide does not have the core-clad structure, Conditional Expressions (5) and (6) are satisfied,

$$0.8 < C / G < 1.5 \qquad ( 5 )$$

$$0.4 < \frac{2 \sqrt{{R}^{2} - {( R - D )}^{2}}}{C} < 0.8 . \qquad ( 6 )$$

### Supplementary Note 4

The illumination optical system according to any one of Supplementary notes 1 to 3,
wherein, in a case where a refractive index of a core with respect to a d line is denoted by Nd1 in a case where the lens closest to the light guide has a core-clad structure in which the core is coated with a clad and a refractive index of the lens closest to the light guide with respect to the d line is denoted by Nd1 in a case where the lens closest to the light guide does not have the core-clad structure, in a case where a distance on the optical axis between the first lens surface and a surface closest to an irradiation target is denoted by L and a focal length of the illumination optical system is denoted by f, Conditional Expression (7) is satisfied,

$$- 1 < L / ( Nd 1 \times f ) < - 0.22 . \qquad ( 7 )$$

### Supplementary Note 5

The illumination optical system according to any one of Supplementary notes 1 to 4, wherein Conditional Expression (1-1) is satisfied,

$$0.7 < \frac{2 \sqrt{{R}^{2} - {( R - D )}^{2}}}{G} < 0.83 . \qquad ( 1 - 1 )$$

### Supplementary Note 6

The illumination optical system according to Supplementary note 2, wherein at least one of Conditional Expression (3-1) or (4-1) is satisfied,

$$0.3 < R / G < 0.5 \qquad ( 3 - 1 )$$

$$2 < \frac{2 \sqrt{{R}^{2} - {( R - D )}^{2}}}{D} < 3.8 . \qquad ( 4 - 1 )$$

### Supplementary Note 7

The illumination optical system according to Supplementary note 3, wherein at least one of Conditional Expression (5-1) or (6-1) is satisfied,

$$1. < C / G < 1.4 \qquad ( 5 - 1 )$$

$$0.45 < \frac{2 \sqrt{{R}^{2} - {( R - D )}^{2}}}{C} < 0.75 . \qquad ( 6 - 1 )$$

### Supplementary Note 8

The illumination optical system according to Supplementary note 4, wherein Conditional Expression (7-1) is satisfied,

$$- 0.5 < L / ( Nd 1 \times f ) < - 0.21 . \qquad ( 7 - 1 )$$

### Supplementary Note 9

An illumination optical system that is disposed at a distal end of a light guide of an endoscope, the illumination optical system comprising:
only two or less lenses as lenses,
wherein the illumination optical system has negative optical power as a whole,
a first lens surface closest to the light guide includes a concave surface that is provided at a center portion thereof and a light diffusion surface that is provided outside the concave surface in a radial direction, and
in a case where an absolute value of a paraxial curvature radius of the concave surface is denoted by R, a distance on an optical axis between an emission end surface of the light guide and the first lens surface is denoted by D, a diameter of the emission end surface of the light guide is denoted by G, and a focal length of the illumination optical system is denoted by f, Conditional Expression (2) is satisfied,

$$0.1 < \frac{2 ( \sqrt{{R}^{2} - {( R - D )}^{2}} ) - G}{f} < 1. \qquad ( 2 )$$

### Supplementary Note 10

The illumination optical system according to Supplementary note 9, wherein Conditional Expressions (3) and (4) are satisfied,

$$0.2 < R / G < 0.6 \qquad ( 3 )$$

$$1.8 < \frac{2 \sqrt{{R}^{2} - {( R - D )}^{2}}}{D} < 4. \qquad ( 4 )$$

### Supplementary Note 11

The illumination optical system according to Supplementary note 9 or 10,
wherein, in a case where a diameter of a core in a direction perpendicular to the optical axis is denoted by C in a case where the lens closest to the light guide has a core-clad structure in which the core is coated with a clad and an outer diameter of the first lens surface is denoted by C in a case where the lens closest to the light guide does not have the core-clad structure, Conditional Expressions (5) and (6) are satisfied,

$$0.8 < C / G < 1.5 \qquad ( 5 )$$

$$0.4 < \frac{2 \sqrt{{R}^{2} - {( R - D )}^{2}}}{C} < 0.8 . \qquad ( 6 )$$

### Supplementary Note 12

The illumination optical system according to any one of Supplementary notes 9 to 11,
wherein, in a case where a refractive index of a core with respect to a d line is denoted by Nd1 in a case where the lens closest to the light guide has a core-clad structure in which the core is coated with a clad and a refractive index of the lens closest to the light guide with respect to the d line is denoted by Nd1 in a case where the lens closest to the light guide does not have the core-clad structure, in a case where a distance on the optical axis between the first lens surface and a surface closest to an irradiation target is denoted by L and a focal length of the illumination optical system is denoted by f, Conditional Expression (7) is satisfied,

$$- 1 < L / ( Nd 1 \times f ) < - 0.22 . \qquad ( 7 )$$

### Supplementary Note 13

The illumination optical system according to Supplementary note 9,
wherein Conditional Expression (2-1) is satisfied,

$$0.2 < \frac{2 ( \sqrt{{R}^{2} - {( R - D )}^{2}} ) - G}{f} < 0.6 . \qquad ( 2 - 1 )$$

### Supplementary Note 14

The illumination optical system according to Supplementary note 10,
wherein at least one of Conditional Expression (3-1) or (4-1) is satisfied,

$$0.3 < R / G < 0.5 \qquad ( 3 - 1 )$$

$$2 < \frac{2 \sqrt{{R}^{2} - {( R - D )}^{2}}}{D} < 3.8 . \qquad ( 4 - 1 )$$

### Supplementary Note 15

The illumination optical system according to Supplementary note 11, wherein at least one of Conditional Expression (5-1) or (6-1) is satisfied,

$$1. < C / G < 1.4 \qquad ( 5 - 1 )$$

$$0.45 < \frac{2 \sqrt{{R}^{2} - {( R - D )}^{2}}}{C} < 0.75 . \qquad ( 6 - 1 )$$

### Supplementary Note 16

The illumination optical system according to Supplementary note 12, wherein Conditional Expression (7-1) is satisfied.

$$- 0.5 < L / ( Nd 1 \times f ) < - 0.21 . \qquad ( 7 - 1 )$$

### Supplementary Note 17

An endoscope comprising:
the illumination optical system according to any one of Supplementary notes 1 to 16.

What is claimed is:

1. An illumination optical system that is disposed at a distal end of a light guide of an endoscope, the illumination optical system comprising only two or less lenses as lenses, wherein:
the illumination optical system has negative optical power as a whole,
a first lens surface closest to the light guide includes a concave surface that is provided at a center portion thereof and a light diffusion surface that is provided outside the concave surface in a radial direction, and
in a case where an absolute value of a paraxial curvature radius of the concave surface is denoted by R, a distance on an optical axis between an emission end surface of the light guide and the first lens surface is denoted by D, and a diameter of the emission end surface of the light guide is denoted by G, Conditional Expression (1) is satisfied,
$$0.6 < \frac{2 \sqrt{{R}^{2} - {( R - D )}^{2}}}{G} < 0.85 . \qquad ( 1 )$$

2. The illumination optical system according to claim 1, wherein Conditional Expressions (3) and (4) are satisfied,
$$0.2 < R / G < 0.6 \qquad ( 3 )$$
$$1.8 < \frac{2 \sqrt{{R}^{2} - {( R - D )}^{2}}}{D} < 4. \qquad ( 4 )$$

3. The illumination optical system according to claim 1, wherein, in a case where a diameter of a core in a direction perpendicular to the optical axis is denoted by C in a case where the lens closest to the light guide has a core-clad structure in which the core is coated with a clad and an outer diameter of the first lens surface is denoted by C in a case where the lens closest to the light guide does not have the core-clad structure, Conditional Expressions (5) and (6) are satisfied,
$$0.8 < C / G < 1.5 \qquad ( 5 )$$
$$0.4 < \frac{2 \sqrt{{R}^{2} - {( R - D )}^{2}}}{C} < 0.8 . \qquad ( 6 )$$

4. The illumination optical system according to claim 1, wherein, in a case where a refractive index of a core with respect to a d line is denoted by Nd1 in a case where the lens closest to the light guide has a core-clad structure in which the core is coated with a clad and a refractive index of the lens closest to the light guide with respect to the d line is denoted by Nd1 in a case where the lens closest to the light guide does not have the core-clad structure, in a case where a distance on the optical axis between the first lens surface and a surface closest to an irradiation target is denoted by L and a focal length of the illumination optical system is denoted by f, Conditional Expression (7) is satisfied,
$$- 1 < L / ( Nd 1 \times f ) < - 0.22 . \qquad ( 7 )$$

5. The illumination optical system according to claim 1, wherein Conditional Expression (1-1) is satisfied,
$$0.7 < \frac{2 \sqrt{{R}^{2} - {( R - D )}^{2}}}{G} < 0.83 . \qquad ( 1 - 1 )$$

6. The illumination optical system according to claim 2, wherein at least one of Conditional Expression (3-1) or (4-1) is satisfied,
$$0.3 < R / G < 0.5 \qquad ( 3 - 1 )$$
$$2 < \frac{2 \sqrt{{R}^{2} - {( R - D )}^{2}}}{D} < 3.8 . \qquad ( 4 - 1 )$$

7. The illumination optical system according to claim 3, wherein at least one of Conditional Expression (5-1) or (6-1) is satisfied,
$$1. < C / G < 1.4 \qquad ( 5 - 1 )$$
$$0.45 < \frac{2 \sqrt{{R}^{2} - {( R - D )}^{2}}}{C} < 0.75 . \qquad ( 6 - 1 )$$

8. The illumination optical system according to claim 4, wherein Conditional Expression (7-1) is satisfied,
$$- 0.5 < L / ( Nd 1 \times f ) < - 0.21 . \qquad ( 7 - 1 )$$

9. An illumination optical system that is disposed at a distal end of a light guide of an endoscope, the illumination optical system comprising: only two or less lenses as lenses, wherein
the illumination optical system has negative optical power as a whole,
a first lens surface closest to the light guide includes a concave surface that is provided at a center portion thereof and a light diffusion surface that is provided outside the concave surface in a radial direction, and
in a case where an absolute value of a paraxial curvature radius of the concave surface is denoted by R, a distance on an optical axis between an emission end surface of the light guide and the first lens surface is denoted by D, a diameter of the emission end surface of the light guide is denoted by G, and a focal length of the illumination optical system is denoted by f, Conditional Expression (2) is satisfied,
$$0.1 < \frac{2 ( \sqrt{{R}^{2} - {( R - D )}^{2}} ) - G}{f} < 1. \qquad ( 2 )$$

10. The illumination optical system according to claim 9, wherein Conditional Expressions (3) and (4) are satisfied,
$$0.2 < R / G < 0.6 \qquad ( 3 )$$
$$1.8 < \frac{2 \sqrt{{R}^{2} - {( R - D )}^{2}}}{D} < 4. \qquad ( 4 )$$

11. The illumination optical system according to claim 9, wherein, in a case where a diameter of a core in a direction perpendicular to the optical axis is denoted by C in a case where the lens closest to the light guide has a core-clad structure in which the core is coated with a clad and an outer diameter of the first lens surface is denoted by C in a case where the lens closest to the light guide does not have the core-clad structure, Conditional Expressions (5) and (6) are satisfied,
$$0.8 < C / G < 1.5 \qquad ( 5 )$$
$$0.4 < \frac{2 \sqrt{{R}^{2} - {( R - D )}^{2}}}{C} < 0.8 . \qquad ( 6 )$$

12. The illumination optical system according to claim 9, wherein, in a case where a refractive index of a core with respect to a d line is denoted by Nd1 in a case where the lens closest to the light guide has a core-clad structure in which the core is coated with a clad and a refractive index of the lens closest to the light guide with respect to the d line is denoted by Nd1 in a case where the lens closest to the light guide does not have the core-clad structure, in a case where a distance on the optical axis between the first lens surface and a surface closest to an irradiation target is denoted by L and a focal length of the illumination optical system is denoted by f, Conditional Expression (7) is satisfied,
$$- 1 < L / ( Nd 1 \times f ) < - 0.22 . \qquad ( 7 )$$

13. The illumination optical system according to claim 9, wherein Conditional Expression (2-1) is satisfied,
$$0.2 < \frac{2 ( \sqrt{{R}^{2} - {( R - D )}^{2}} ) - G}{f} < 0.6 . \qquad ( 2 - 1 )$$

14. The illumination optical system according to claim 10, wherein at least one of Conditional Expression (3-1) or (4-1) is satisfied,
$$0.3 < R / G < 0.5 \qquad ( 3 - 1 )$$
$$2 < \frac{2 \sqrt{{R}^{2} - {( R - D )}^{2}}}{D} < 3.8 . \qquad ( 4 - 1 )$$

15. The illumination optical system according to claim 11, wherein at least one of Conditional Expression (5-1) or (6-1) is satisfied,
$$1. < C / G < 1.4 \qquad ( 5 - 1 )$$
$$0.45 < \frac{2 \sqrt{{R}^{2} - {( R - D )}^{2}}}{C} < 0.75 . \qquad ( 6 - 1 )$$

16. The illumination optical system according to claim 12, wherein Conditional Expression (7-1) is satisfied.
$$- 0.5 < L / ( Nd 1 \times f ) < - 0.21 . \qquad ( 7 - 1 )$$

17. An endoscope comprising the illumination optical system according to claim 1.

18. An endoscope comprising the illumination optical system according to claim 9.
